(19) United States
(12) Patent Application Publication
(10) Pub. No.: US 2005/0054722 A1
(43) Pub. Date: Mar. 10, 2005

(54) Inhibition of two-component signal transduction systems

(75) Inventors: Dacre England, Raudwick (AU); Staffan Kjelleberg, La Perouse (AU)

Correspondence Address: NIXON & VANDERHYE, PC, 1100 N GLEBE ROAD, 8TH FLOOR, ARLINGTON, VA, 22201-4714, US

(73) Assignee: Unisearch Limited,, Sydney (AU)

(21) Appl. No.: 10/954,288

(22) Filed: Oct. 1, 2004

Related U.S. Application Data
(63) Continuation of application No. 10/168,141, filed on Jul. 2, 2002.

(30) Foreign Application Priority Data
Dec. 17, 1999 (AU) PQ 4755

(51) Int. Cl. A61K 31/22; A61K 31/365; A61K 31/12; A61K 31/11

(52) U.S. Cl. 514/473; 514/546; 514/693; 514/675

(57) ABSTRACT

The present invention provides compositions and methods for inhibition activities and actions of microorganisms, particularly bacteria. The compositions and methods are based primarily on the inhibition of two-component signal transduction systems with halogenated furanones and related 3-haloalkenones.

### FIELD OF THE INVENTION

The present invention is directed generally to compositions and methods for inhibition of activities and actions of microorganisms, particularly inhibition of two-component signal transduction systems.

### BACKGROUND OF THE INVENTION

Two-component signal transduction systems play important roles in the growth and maintenance and functionality of many different microorganisms. Examples include, but not are limited to, regulation of the production of exopolysaccharides and virulence factors; the regulation of motility, swarming, attachment and biofilm formation; and growth and maintenance of viability.

There have been a limited number of reports of inhibitors of two-component signal transduction systems. Roychoudhury and co-workers (1993) screened a large bank of compounds in an assay that determined the activity of the AlgR₂/AlgR₁ system in Pseudomonas aeruginosa by measuring the transcription of a plasmid borne algD-xylE fusion. The AlgR₂AlgR₁ two-component system plays a role in regulating the production of the exopolysaccharide alginate (Deretic et al., 1989). Of the 25,000 compounds screened, two classes were identified that significantly inhibited transcription of the algD-xylE fusion. Among these where Inhibitor A, belonging to a class of isothiazalones, and Inhibitor B, a member of the quaternary imidazoles. Inhibitor A was shown to inhibit the autophosphorylation of the histidine protein kinase (HPK) AlgR₂. Inhibitor B interfered with the binding of the response regulator (RR) AlgR₁, in its phosphorylated form, to its target DNA promoter site, as determined in a gel mobility shift assay. The authors did not indicate whether the compounds reduced in vivo alginate production or had any antibacterial activity. More recently, Ulijasz and Weisblum (1999) carried out further in vitro experiments with Inhibitor A and the VanS/VanR system which controls inducible vancomycin resistance in Enterococcus faecium (Arthur et al., 1992). This study demonstrated that inhibitor A inhibits the phosphoryl transfer from the phosphorylated form of the VanS HPK to its coupled response regulator VanR in vitro. The authors concluded that inhibitor A was acting on the response regulator VanR in such a way that it blocked phosphoryl transfer from VanS to VanR. This finding conflicts with those of Roychoudhury et al., (1993), where inhibitor A was shown to inhibit autophosphorylation of the HPK AlgR₂.

Domagala et al. (1998) have identified another class of inhibitors of two-component signal transduction systems. This group screened for compounds that could de-phosphorylate the soluble HPK NRII in vitro, and identified a number of diphenolic methanes which showed significant activity. The compounds were also tested against two-component systems in vivo using Escherichia coli and were demonstrated to be active. The assays used were of the authors' devising and were not described in great detail.

Those diphenolic methanes that appeared most active against two-component signal transduction systems were tested for antibacterial activity and inhibited the growth of a number of Gram positive organisms, including Bacillus subtilis, Staphylococcus aureus, Enterococcus faecium and Streptococcus pyogenes. Interestingly, drug resistant strains of both E. faecium and S. aureus remained sensitive. The Gram negative bacterium E. coli was not sensitive but a cell wall permeable (imp minus) strain. E. coli LKY, had sensitivity approaching that of the various Gram positive organisms. The compounds were also found to have a second mode of action, that of membrane perturbation, which was determined using propidium iodide uptake experiments.

Barrett et al. (1998) showed that a family of hydrophobic tyramines could interfere with the normal function of two-component signal transduction systems. The most potent of these compounds, was designated RWJ-49815. The authors demonstrated that this family of compounds inhibited the autophosphorylation of the purified HPK KinA of B. subtilis, and also showed that these compounds interfered with the normal activity of the in vivo Taz/OmpR two-component assay of Jin and Inouye (1993) described below.

RWJ-49815 and its analogues also proved to be potent Gram positive antibacterial compounds, active at concentrations of 1-2 μg/ml against S. aureus, E. faecium and Streptococcus pneumoniae.

A second paper published by members of the same laboratory identified a further class of inhibitors of two-component systems, the substituted salicyanilides (Maclielag et al., 1998). In vitro tests using KinA and its RR partner SpoOF showed that these compounds inhibited the autophosphorylation of KinA. The authors also made use of an in vivo assay for two-component signal transduction based on the VanS/VanR system. The salicyanilides had antibacterial effects against Gram positive organisms but had no effect on wild type E. coli. However, a mutant E. coli strain possessing a leaky outer membrane was as sensitive to the compounds as any of the Gram positive organisms tested.

Hilliard et al., (1999) showed that both these families of compounds, tyramines and salicyanilides, have more mechanisms of action than just inhibition of two-component signal transduction systems. While the authors were able to show that RWJ-49815 inhibited the autophosphorylation of the HPK NRII, the compound also caused a rapid increase in the permeability of the membranes of S. aureus cells as determined by propidium iodide staining. Furthermore, the compounds triggered the rapid and complete lysis of equine erythrocytes. The salycylanides caused little membrane damage and significantly less haemolysis, but there was no correlation between their inhibitory effects on the autophosphorylation of HPKs KinA and NRII and their antibacterial activity against Gram positives.

Fabret and Hoch (1998) identified a response regulator, YycF, in Bacillus subtilis that is required for this organism's growth. When a thermosensitive mutant of YycF is grown at a nonpermissive temperature, growth rapidly ceases and empty cells are formed that retain their structural integrity. YycF belongs to the OmpR winged helix-turn-helix family of DNA-binding proteins and has a paired histidine protein kinase, YycG. Both members of this two-component signal transduction system are transcribed throughout the growth phase of B. subtilis but are not transcribed in stationary phase.

Martin et al. (1999) identified a homologous two-component signal transduction system in Staphylococcus aureus that is also required for growth. The authors could not generate a YycF knock out, but, like Fabret and Hoch (1998), managed to generate a thermosensitive mutant strain with which they could determine that the YycG/YycF system is involved in controlling cell permeability.

Lange et al. (1999) have identified a YycG/YycF two-component signal transduction system in Streptococcus pneumoniae that is also required for growth and there are YycG/YycF homologues in the genomes of a least two further Gram positives, Enterococcus faecalis and Streptococcus pyrogenes. The genome of Lactococcus lactis also possesses a yycF homologue but the genome does not appear to possess the pair histidine protein kinase YycG (Bolotin et al., 1999). It is possible that these homologous and perhaps indispensable two-component signal transduction systems are one important target for the antibacterial compounds described above.

The diphenolic methanes, hydrophobic tyramines and substituted salicyanilides have inhibitory effects on the in vivo activity of two-component signal transduction systems and also have strong growth inhibitory activity against Gram positives while having little effect on Gram negatives with intact outer membranes (Domagala et al., 1998; Barrett et al., 1998; Macielag et al., 1998).

### SUMMARY OF THE INVENTION

In a first aspect the present invention consists in a composition for use in inhibiting at least one phenotype of a microorganism, the composition comprising at least one compound of general formula I: 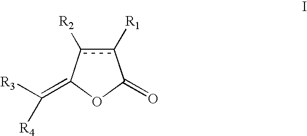
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy, alkylsilyl;
R₃ or R₄+R₂ can be a saturated or an unsaturated cycloalkane;
and 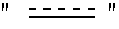
represents a single bond or a double bond provided that at least one of R₁, R₂, R₃ and R₄ is halogen and where R₃=H and R₄=Ph, R₁ and R₂ can independently be H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
or a compound of general formula II 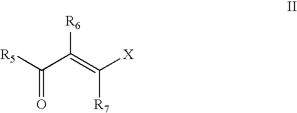
wherein R₆ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
X is a halogen;
R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

In a second aspect the present invention consists in a method of inhibiting at least one phenotype of a microorganism, the method comprising exposing the microorganism to a composition comprising at least one compound of general formula I: 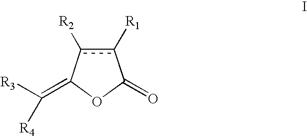
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy, alkylsilyl;
R₃ or R₄+R₂ can be a saturated or an unsaturated cycloalkane;
and 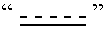
represents a single bond or a double bond provided that at least one of R₁, R₂, R₃ and R₄ is halogen and where R₃=H and R₄=Ph, R₁ and R₂ can independently be H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
or a compound of general formula II 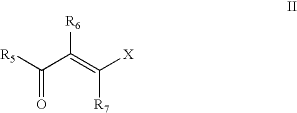
wherein R₈ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic,
X is a halogen;
R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

The term “alkyl” is taken to mean both straight chain alkyl groups such as methyl, ethyl, propyl, isopropyl, butyl, isobutyl, sec-butyl, tertiary butyl, and the like. Preferably the alkyl group is a lower alkyl of 1 to 6 carbon atoms. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, halohetorocyclyl, alkylsulfenyl, alkylcarbonyloxy, alkylthio, ecylthio, phosphorus-containing groups such as phosphono and phosphinyl.

The term “alkoxy” denotes straight chain or branched alkyloxy, preferably C₁₋₁₀ alkoxy. Examples include methoxy, ethoxy, n-propoxy, isopropoxy and the different butoxy isomers.

The term “alkenyl” denotes groups formed from straight chain, branched or mono- or polycyclic alkenes and polyene. Substituents include mono- or poly-unsaturated alkyl or cycloalkyl groups as previously defined, preferably C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butedienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

The term “halogen” denotes fluorine, chlorine, bromine or iodine, preferably bromine or fluorine.

The term “heteroatoms” denotes O, N or S.

The term “acyl” used either alone or in compound words such as “acyloxy”, “acylthio”, “acylamino” or “diacylamino” denotes an aliphatic acyl group and an acyl group containing a heterocyclic ring which is referred to as heterocyclic acyl, preferably a C₁₋₁₀ alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl, heterocycloalkanecarbonyl; heterocyclyoalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

As will recognised by those skilled in the art the compounds of general formulas II, III and IV can exist as two isomers e and z. It is intended that the general formulas depicted herein are not limited to a particular isomer and encompass both isomers either in the form of a racemic mixture or separated isomers.

In a preferred embodiment the phenotype is controlled by a two-component signal transduction system. Preferably, the two-component signal transduction system is selected from, but not limited to, those whose response regulator belongs to the FixJ/LuxR subfamily or the OmpR subfamily of response regulators.

It is preferred that the phenotype of the microorganism is selected from the group consisting of growth, swarming/motility, biofilm formation, expression of virulence factors and combinations thereof.

In a preferred embodiment of the present invention the microorganism is selected from the group consisting of Bacillus sp. Streptococcus sp., Helicobacter sp., Mycobacterium sp, Staphylococcus sp, Enterobacter sp. Pseudomonas sp., and Bordatella sp. In particular it is preferred that the microorganism is selected from the group consisting of Bacillus subtilis, Bacillus anthracis, Bacillus cereus, Bacillus licheniformus, Streptococcus pneumonia, Helicobacter pylori, Mycobacterium tuberculosis, Staphylococcus aureus, Staphylococcus epidermis, Enterobacter faecalis, Pseudomonas syringae, Pseudomonas aeruginosa, and Bordatella pertusis.

In a further preferred embodiment the composition comprises at least one compound selected from the group consisting of compounds 2, 3, 4, 30, 33, 34, 80, 97 as set out in Table 1 and combinations thereof.

In a third aspect the present invention consists in a method of preventing or reducing biofilm formation on a surface, the method comprising applying to the surface the composition of the first aspect of the present invention.

In a fourth aspect the present invention consists in a method of treating bacterial infection or decreasing the severity of symptoms of bacterial infection in an animal, the method comprising administering to the animal an effective amount of the composition of the first aspect of the present invention.

The composition of the present invention can be used in environmental, sanitary, veterinary, or medical applications where it is possible to effect the phenotype of a microorganism, particularly through inhibition of a two-component signal transduction system A particular two-component signal transduction system maybe targeted by use or selection of the compound or mixture of compounds. Similarly, a particular microorganism may be targeted by use or selection of the compound or mixture of compounds.

Applications include, but are not limited to, inhibition of growth of microbial pathogens in environmental situations, reduction or prevention of microbial colonisation of medical media including washing solutions, ointments and the like, inhibition of microbial attachment to surfaces and subsequent biofilm formation, as active ingredients in antiseptics and disinfectants.

As will be recognised by those skilled in the art the compounds of formulae I and II can be usefully incorporated in a varied range of compositions. For example the compounds can be incorporated in a range of personal care products such as deodorants, soaps, shampoos, dentifrices etc. The manufacture of such compositions is well known in the art and the compounds of formulae I and II or mixtures thereof can be simply included in these compositions in admixture.

The ability of compositions comprising the compounds of formulae I and II or mixtures to inhibit phenotypes of a range of bacteria provides a number of useful applications of these compositions. In particular the compositions may be formulated for pharmaceutical use with human and non-human animals. In one embodiment of the invention the compositions are formulated for topical application for use, for example, in application to wounds and the like. In this regard they may be directly incorporated into bandages and the like.

The compositions of the present invention will also find application in preventing or inhibiting biofilm formation. In another embodiment the compositions will find application as washing solutions, particularly in contact lens cleaning compositions.

It has been found by the present inventors that with a number of the compounds a concentration of less than 25 μg/ml in vivo is sufficient to inhibit the normal function of a number of two-component signal transduction systems. It will be appreciated, however, that the concentration required may depend on a number of factors including the microorganism, the furanone compound(s) used, the two-component signal transduction system to be inhibited, and the formulation of the furanone into the product.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### DETAILED DESCRIPTION

In order that the present invention may be more clearly understood, preferred forms will be described with reference to the following non-limiting examples and drawings.

Figure Legend

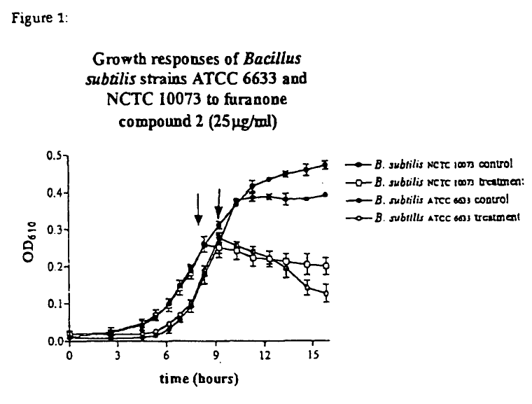

FIG. 1 shows the growth responses of Bacillus subtilis strain ATCC 6633 and NCTC 10073 to compound 2. The compound was added at 8-9 hours, as denoted by the arrows, after the cultures had been growing. B. subtilis has a two component system that, when deleted, results in lysis and cell death. Addition of the compounds to B. subtilis also results in the induction of cell lysis, which can be observed as a cessation of growth and even a decrease in optical density. Therefore, this data suggests that the compounds interfere with this two component system and cause cell death or prevent growth.

Bacterial Strains and Plasmids

The bacterial strains and plasmids used in the following Examples are set out in Table 2.

Two-Component Signal Transduction Assays

Taz-1 Assay

The Taz-assay carried out according to the method of Jin and Inouye (1993) with the following alterations. E. coli RU1012 (pYT0301) were grown overnight in M9 medium at 37° C. supplemented with 100 μg/ml ampicillin and 50 μg/ml kanamycin. This overnight culture was then used to inoculate 50 ml M9 medium in side-arm flasks which were then incubated at 37° C. and shaken at 180 rpm. The OD₆₁₀ of the growing cultures was monitored regularly and when the OD₆₁₀=0.2 the cultures were placed on ice. Aspartate was added to side-arm flasks to give a final concentration of 3 mM (aspartate stock solution made up in M9 salts).

The test compound or mixtures of compounds were dissolved in ethanol and added to cultures to give the required final concentrations. Negative controls were prepared with equal volumes of ethanol. Cultures were then placed in a 37° C. incubator and shaken for 4 hours (OD₆₁₀ approximately 0.7) before being removed and put on ice. Samples were then removed for β-galactosidase assays carried out according to the method of Miller (1972).

The results obtained in this assay are set out in Table 3.

CopS/CopR Assay

P. syringae pv syringae PS61 (pCOP38)(pPT23D) was grown on SWM media (Kinscherf and Willis, 1999) at room temperature with shaking for 48 hours. Five μg/ml streptomycin, 15 μg/ml chloramphenicol and 1.0 mM CuSO₄ were added to maintain plasmids. This culture was used to inoculate 50 ml SWM media in side-arm flasks with the addition of antibiotics. These cultures were incubated at room temperature with shaking for 16 hours (OD₆₁₀=0.2) at which point CuSO₄ was added to a concentration of 0.075 mM (CuSO₄ solution made up in MQ water).

The test compound or mixtures of compounds were dissolved in ethanol and added to cultures to give the required final concentrations. Equal volumes of ethanol were added to Cu²⁺ negative and positive cultures. Cultures were incubated for 6.5 hours at room temperature with shaking before being placed on ice. Samples were then removed for β-galactosidase assays. β-galactosidase assays were carried out in the same manner as those for the Taz assay described above.

The effect of furanone compound 3 on the CopS/CopR two component signal transduction system that regulates copper resistance in Pseudomonas syringae pv. syringae (Mills et al., 1993) was assessed. Compound 3 at concentrations of 25 μg/ml and 50 μg/ml significantly reduced cop′-lacZ expression (p>0.05). However, there appears to be no difference in terms of lacZ expression between the two concentrations (p>0.15). Compound 3 did not have any growth inhibitory effects at the concentrations used Compound 4 also appeared to reduce the normal activity of the CopS/CopR two-component signal transduction system.

GacS/GacA Assay

P. syringae var tomato BB27 was grown overnight in SWM media at room temperature. This culture was used to stab inoculate SWM plates made up with 0.4% agar and incubated at room temperature (20° C.). The culture was also used to stab inoculate sets of SWM plates (0.4% agar) that bad been made up with 25 μg/ml and 50 μg/ml of the test compound (stock solutions made up in ethanol). These plates were also incubated at room temperature for 36 hours before being examined for swarming activity and photographed. Before use all 0.4% agar SWM plates were allowed to air-dry for two hours in a laminar flow cabinet at room temperature.

Furanones interfere with the “swarming” response of Pseudomonas syringae, which is regulated by the GacS/GacA two-component signal transduction system (Kinscherf and Willis, 1999). Furanone compound 3 was found to shut down swarming at 50 μg/ml and dramatically alters the swarming pattern at a concentration of 25 μg/ml. Compound 3 did not inhibit the growth of P. syringae var. tomato at a concentration of 50 μg/ml. Furanone compound 30 also inhibited the swarming response in P. syringae.

Growth Curves

Growth curve method. Bacteria are grown overnight in standard medium. The following morning, the cells were inoculated into fresh medium at 1% (a 1 in 100 dilution). Furanones were added either at the beginning of growth (time 0) or, as was the case for the B. subtilis experiments, the results of which are shown in FIG. 1, during the mid-logarithmic phase of growth. Growth was then monitored regularly by spectrophotometric readings, at a wavelength of 610 nm.

MIC's for Staphylococcus aureus

Using the type of growth described above, the minimum growth inhibitory concentration of furanones was determined for S. aureus and Streptococcus spp. were determined for a range of compounds. The results are set out in Table 4.

Without wishing to be bound by scientific theory it would appear from the data presented above that the compounds and mixtures thereof interfere with the normal function of a number of two-component signal transduction systems:
the compounds shut down signal transduction triggered by aspartate in the Taz-assay;
reduce the degree of signal transduction triggered by Cu²⁺ ions in the CopS/CopR assay; and
appear to modulate swarming in P. syringae that is known to be, at least in part, regulated by the GacS/GacA two-component signal transduction system.
   Furanones as Inhibitors of Signal Transduction Systems: Effects on the Colonisation of Surfaces

Given that the furanones and related compounds of the present invention interfere with the normal function of two-component signal transduction systems, it may be that the furanones block the attachment of bacteria to the surface, by interfering with one or more of these systems.

There is certainly some evidence that two-component signal transduction systems play a central role in the attachment of bacteria to surfaces. For example, the ColS/ColR two-component signal transduction system in Pseudomonas fluorescens strain WCS365 plays an important role in the attachment of this bacterial strain to root surfaces (Dekkers et al., 1998). A mutant stain with a colS/colR deletion colonises root surfaces up to 1,000 fold less efficiently than a wild-type strain. This reduced ability to attach to a surface could not be ascribed to any defects in chemotaxis, motility or a reduced ability to take up a range of plant exudates. No gene or set of genes has yet been found that is regulated by this two-component signal transduction system, nor do the identities of ColR and ColS's closest characterised homologues, which include CopR_{P. syringae} (61% similarity and 38.5% identity) in the case of ColR and CpxA_{E. coli} (53% similarity and 26% identity) in the case of ColS, indicate what phenotypes) this two-component system regulate.

Recently Philippe Lejeune and colleagues have shown that two-component signal transduction systems play an important role in the attachment of E. coli to abiotic surfaces. Firstly, it was demonstrated that the EnvZ/OmpR two-component system was important for attachment and subsequent biofilm formation (Vidal et al., 1998). It was shown that OmpR controls the production of the curli by directly regulating the expression of csgA, which encodes one of the major components of curli. Curli appear to be absolutely required for attachment and biofilm formation by E. coli for both characterised laboratory strains and a limited number of clinical isolates (Vidal et al., 1998; Dorel et al., 1999). Secondly, the CpxA/CpxR two-component system similarly regulates the expression of the csgA, thereby controlling the number of curli produced (Dorel et al., 1999). Other groups have demonstrated that structures on the surface of E. coli are important for attachment, for example Pratt and Kolter (1998) demonstrated that type I pili are required for E. coli strains to permanently attach to a surface, and it is likely that two-component signal transduction systems play some role in their regulation.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

TABLE 1

| Compound | |
|---|---|
| No. | Structure |
| 2 (d3) | 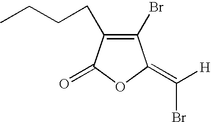 |
| 3 (d5) | 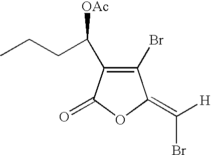 |
| 4 (d19) | 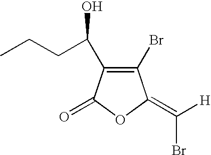 |
| 30 | 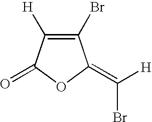 |
| 33 | 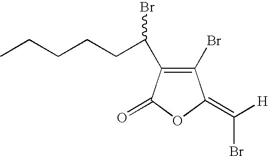 |
| 34 | 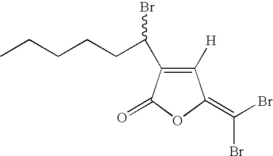 |
| 75 | 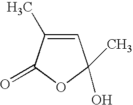 |
| 76 | 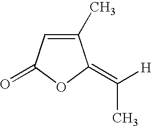 |
| 80 | 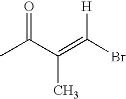 |
| 92 | 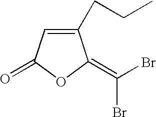 |
| 96 | 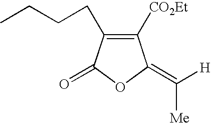 |
| 97 | 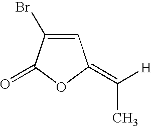 |
| A19 | 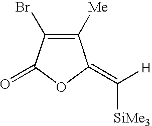 |

TABLE 2

| Strain | Genotype | Reference |
|---|---|---|
| Escherichia coli | Ø(ompC-lacZ)10-25, | Utsumi et al., 1989 |
| RU1012 | ΔenyZ::Km^{R} | |
| E. coli RC11/ | ΔnarXL, ΔnarQ::Km^{R}, | Cavicchioli et al., 1995 |
| λLK1 | recA56, λPC51 | |
| | Ø(narG-lacZ) | |
| P. syringae pv. | Rif^{S}, Cam^{R}, Cu^{S} | Bender & Cooksey, 1986 |
| syringae PS61 | | |
| P. syringae var. | wild-type | UNSW Culture Collection |
| tomato BB27 | | |
| Plasmids | Description | Reference |
| pYT0301 | tar-envZ (Taz-1), Amp^{R} | Yang & Inouye, 1991 |
| pLK63 | narX⁺, narL⁻, Cm^{R} | Kalman & Gunsalus, 1989 |
| pCOP38 | Sm^{R}, Cm^{R}, pMP190 | Meliano & Cooksey, 1988 |
| | with coplacZ | |
| | promoter fusion | |
| pPT23D | Cu^{R}, wild-type plasmid | Bender & Cooksey, 1986 |
| | carrying cop operon and | |
| | copSicopR | |

TABLE 3

| Treatment | Percentage Induction |
|---|---|
| C2 15 μg/ml | 34.6 |
| C2 25 μg/ml | 15.0 |
| Negative Control | 23.7 |
| C3 25 μg/ml | 27.0 |
| Negative Control | 25.5 |
| C30 5.0 μg/ml | 36.9 |
| C34 2.5 μg/ml | 76.1 |
| Negative Control | 17.2 |
| C30 1.25 μg/ml | 52.3 |
| C30 2.50 μg/ml | 34.5 |
| C34 1.25 μg/ml | 92.8 |
| C34 2.50 μg/ml | 84.0 |
| Negative Control | 8.5 |
| C75 25 μg/ml | 122.6 |
| C76 25 μg/ml | 111.2 |
| Negative Control | 5.8 |
| C80 25 μg/ml | 71.6 |
| AI9 0.5 μg/ml | 113.4 |
| AI9 1.0 μg/ml | 94.2 |
| Negative Control | 17.8 |

TABLE 4
Minimum inhibitory (growth) concentrations of furanones

| Compound | Staphylococcus aureus | Streptococcus spp. |
|---|---|---|
| 2 | 1-10 ug/ml | Not Tested |
| 3 | Not tested | Not tested |
| 4 | Not Tested | 10 ug/ml |
| 30 | 1-20 ug/ml | 10 ug/ml |
| 33 | 500 ng/ml | Not tested |
| 34 | 250 ng/ml | Not tested |
| 33/34 | 1 ug/ml | 10 ug/ml |
| 45 | 1-20 ug/ml | 10 ug/ml |
| 80 | Not Tested | Not tested |
| 97 | Not Tested | Not tested |

### REFERENCES

Arthur, M., C Molinas, and P Courvalin 1992. The VanS-VanR two-component regulatory system controls synthesis of depsipeptide peptidoglycan precursors in Enterococcus faecium BM4147. J. Bacteriol. 174: 2582-2591
Barrett J. F., R. M. Goldschmidt, L. E. Lawrence, B. Foleno, R. Chen, J. P. Demers, S. Johnson, R. Kanojia, J. Fernandez, J. Bernstein, L. Licata, A. Donetz, S. Huang, D. J. Hlasta, M. J. Macielag, K. Ohemeng, R. Frechette, M. B. Frosco, D. H. Klaubert, J. M. Whiteley, L. Wang, and J. A. Hoch 1998. Antibacterial agents that inhibit two-component signal transduction systems. Proc. Natl. Acad. Sci. USA 95: 5317-5322
Bender, C. L., and D. A. Cooksey 1986. Indigenous plasmids in Pseudomonas syringae pv. tomato: conjugative transfer and role in copper resistance. J. Bacteriol. 165: 534-541
Bolotin, A., S. Mauger, K. Malarme, S. D. Ehrlich and A. Sorokin 1999. Low-redundancy sequencing of the entire Lactococcus lactis IL1403 genome. Antonie van Leeuwenhoek 76: 27-76
Cavicchioli, R., I Schroder, M Constanti, and R P Gunsalus 1995. The NarX and NarQ sensor-transmitter proteins of Escherichia coli each require two conserved histidines for nitrate-dependent signal transduction to NarL. J. Bacteriol. 177: 2416-2424
Dekkers, L. C, C. J. P. Bloemendaal, L. A. de Weger, C. A. Wijffelman, H. P. Spaink and B. J. J. Lugtenberg 1998. A two-component system plays an important role in the root-colonizing ability of Pseudomonas fluorescens strain WCS365 Molecular Plant-Microbe Interactions 11(1) 45-56
Deretic, V., R. Kishit, W. M. Konyesni, A. M. Chakrabarty and T. K. Misra 1989. The algR gene, which regulates mucoidy in Pseudomonas aeruginosa, belongs to a class of environmentally responsive genes. J. Bacteriol. 171: 1278-1283
Dorel, C., O. Vidal, C. Prigent-Combaret, I. Vallet and P. Lejeune 1999. Involvement of the Cpx signal transduction pathway in E. coli in biofilm formation. FEMS Microbiology Letters 178: 169-175
Fabret, C., and J. A. Hoch 1998. A two-component signal transduction system essential for growth of Bacillus subtilis: implications for anti-infective therapy. J. Bacteriol. 180: 6375-6383
Hilliard, J. J., R. M. Goldschmidt, L. Licata, E. Z. Baum, and K. Bush 1999. Multiple mechanisms of action for inhibitors of histidine protein kinases from bacterial two-component systems. Antimicrobial Agents and Chemotherapy 43: 1693-1699
Jin, T., and M. Inouye, 1993. Ligand binding to the receptor domain regulates the ratio of kinase to phosphatase activities of the signalling domain of the hybrid Escherichia coli transmembrane receptor, Tazl. J. Mol. Biol. 232: 484-492
Kalman, L. V., and R. P. Gunsalus 1989. Identification of a second gene involved in global regulation of fumurate reductase and other nitrate-controlled genes for anaerobic respiration in Escherichia coli. J. Bacteriol. 171: 3810-3816
Kinscherf, T. G., and D. K. Willis 1999. Swarming by Pseudomonas syringae B728a requires gacS (lemA) and gacA but not the acyl-homoserine lactone biosynthetic gene ahlI. J. Bacteriol. 181: 4133-4136
Lange, R., C. Wagner, A. de Saizieu, N. Flint, J. Molnos. M. Stieger, P. Caspers, M. Kamber, W. Keck and K. E. Amrein 1999. Domain organisation and molecular characterization of 13 two-component systems identified by genome sequencing of Streptococcus pneumoniae. Gene 237: 223-234
Macielag, M. J., J. P. Demers, S. A. Fraga-Spano, D. J. Hlasta, S. G. Johnson, R. M. Kanojiia, R. K. Russell, Z. Sui, M. A. Weidner-Wells, H. Werblood, B. D. Foleno. R. M. Goldschmidt, M. J. Loeloff, G. C. Webb, and J F. Barrett 1998.
Substituted salicylanilides as inhibitors of two-component regulatory systems in bacteria. J. Med. Chem. 41: 2939-2945
Martin, P. K., T. Li, D. Sun, D. P. Biek, and M. B. Schmid 1999. Role in cell permeability of an essential two-component system in Staphylococcus aureus. J. Bacteriol 181: 3666-3673
Mellano, M. A., and D. A. Cooksey 1988. Induction of the copper resistance operon from Pseudomonas syringae pv. tomato. J. Bacteriol 170: 4399-4401
Mills, S. D., C. A. Jasalavich and D. A. Cooksey 1993. A two-component system required for copper-inducible expression of the copper resistance operon of Pseudomonas syringae. J. Bacteriol. 175: 1656-1664
Pratt, L. A., and R. Kolter 1998. Genetic analysis of Escherichia coli biofilm formation: roles of flagella, motility, chemotaxis and type I pili. Mol. Microbiol. 30: 285-293
Roychoudhury, S., N. A. Zielinski, A. J. Ninfa. N. E. Allen, L. N. Jungheim, T. I. Nicas, and A. M. Chakrabarty. 1993. Inhibitors of two-component signal transduction systems: inhibition of alginate gene activation in Pseudomonas aeruginosa. Proc. Natl. Acad. Sci. USA 90: 985-969
Ulijasz, A. T., and B. Weisblum 1999. Dissecting the VanRS signal transduction pathway with specific inhibitors. J. Bacteriol. 181: 627-631
Utsumi, R., R. E. Brissette, A. Rampersaud, S. A. Forst. K. Oosawa and M. Inouye 1989. Activation of bacterial porin gene expression by a chimeric signal transducer in response to aspartate. Science 245: 1246-1249
Vidal, O., R. Longin, C. Prigent-Combaret, C. Dorel, M. Hooreman and P. Lejeune 1998. Isolation of an Escherichia coli K-12 mutant strain able to form biofilms on inert surfaces: involvement of a new ompR allele that increases curli expression. J. Bacteriol. 180(9): 2442-2449
Yang, Y., and M. Inouye 1991. Intermolecular complementation between two defective mutant signal transducing receptors of Escherichia coli. Proc. Natl. Acad. Sci. USA 88: 11057-11061

1. A composition for use in inhibiting at least one phenotype of a microorganism, the composition comprising at least one compound of general formula I: 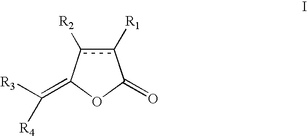
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy, alkylsilyl;
R₃ or R₄+R₂ can be a saturated or an unsaturated cycloalkane; and 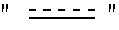
represents a single bond or a double bond provided that at least one of R₁, R₂, R₃ and R₄ is halogen and where R₃=H and R₄=Ph, R₁ and R₂ can independently be H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
or a compound of general formula II 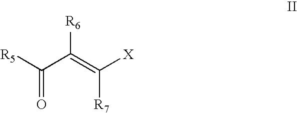
wherein R₆ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
X is a halogen;
R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

2. A composition as claimed in claim 1 in which the phenotype is controlled by a two-component signal transduction system.

3. A composition as claimed in claim 1 in which the phenotype of the microorganism is selected from the group consisting of growth, swarming/motility, expression of virulence factors and combinations thereof.

4. A composition as claimed in claim 1 in which the microorganism is selected from the group consisting of Bacillus sp., Streptococcus sp., Helicobacter sp., Mycobacterium sp, Staphylococcus sp, Enterobacter sp., Pseudomonas sp., and Bordatella sp.

5. A composition as claimed in claim 4 in which the microorganism is selected from the group consisting of Bacillus subtilis, Bacillus anthracis, Bacillus cereus, Bacillus licheniformus, Streptococcus pneumonia, Helicobacter pylori, Mycobacterium tuberculosis, Staphylococcus aureus, Staphylococcus epidermis, Enterobacter faecalis, Pseudomonas syringae, Pseudomonas aeruginosa, and Bordatella pertusis.

6. A composition as claimed in claim 1 in which the composition comprises a pharmaceutically acceptable carrier or excipient.

7. A composition as claimed in claim 1 in which the composition is a dentifrice.

8. A composition as claimed in claim 1 in which the composition is a deodorant.

9. A composition as claimed in claim 1 in which the composition is a cleaning composition.

10. A composition as claimed in claim 1 in which the composition is a hair cleaning composition.

11. A composition as claimed in claim 1 in which the composition is a contact lens cleaning composition.

12. A composition as claimed in claim 1 in which the composition is a soap.

13. A composition as claimed in claim 6 in which the composition is formulated for topical administration.

14. A composition as claimed in claim 6 in which the composition is applied to a bandage.

15. A composition as claimed in claim 1 in which the composition comprises at least one compound selected from the group consisting 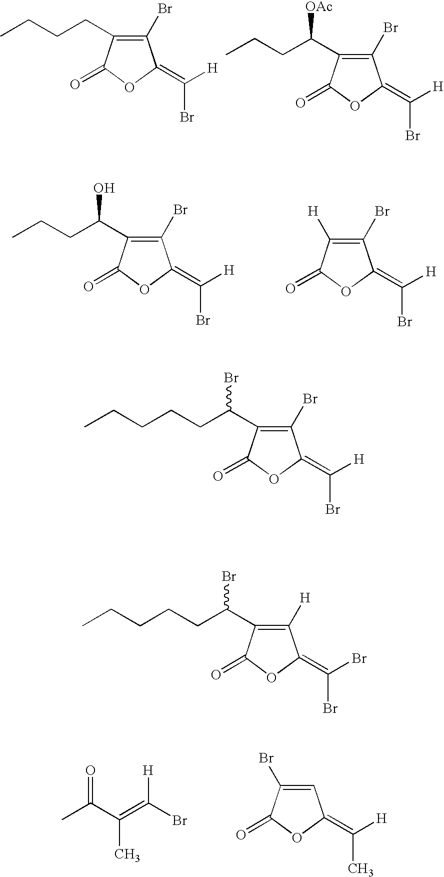
and combinations thereof.

16. A method of inhibiting at least one phenotype of a microorganism, the method comprising exposing the microorganism to a composition comprising at least one compound of general formula I: 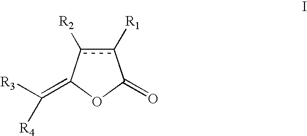
wherein R₁ and R₂ are independently H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
R₃ and R₄ are independently H, halogen, alkyl, aryl or arylalkyl, alkoxy, alkylsilyl,
R₃ or R₄+R₂ can be a saturated or an unsaturated cycloalkane; and 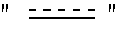
represents a single bond or a double bond provided that at least one of R₁, R₂, R₃ and R₄ is halogen and where R3=H and R4=Ph, R1 and R2 can independently be H, halogen, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
or a compound of general formula II 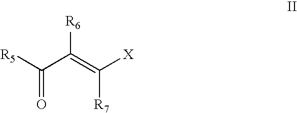
wherein R₆ and R₇ are independently H, halogen, carboxyl, ester, formyl, cyano, alkyl, alkoxy, oxoalkyl, alkenyl, aryl or arylalkyl whether unsubstituted or substituted, optionally interrupted by one or more heteroatoms, straight chain or branched chain, hydrophilic or fluorophilic;
X is a halogen;
R₅ is H, alkyl, alkenyl, alkynyl, alkene, alkyne, aryl, arylalkyl, whether unsubstituted or substituted, optionally interrupted by one or more heteroatoms, straight chain or branched chain, hydrophilic or fluorophilic.

17. A method as claimed in claim 16 in which the phenotype is controlled by a two-component signal transduction system.

18. A method as claimed in claim 16 in which the phenotype of the microorganism is selected from the group consisting of growth, swarming/motility, expression of virulence factors and combinations thereof.

19. A method as claimed in claim 16 in which the microorganism is selected from the group consisting of Bacillus sp., Streptococcus sp., Helicobacter sp., Mycobacterium sp, Staphylococcus sp, Enterobacter sp., Pseudomonas sp., and Bordatella sp.

20. A method as claimed in claim 19 in which the microorganism is selected from the group consisting of Bacillus subtilis, Bacillus anthracis, Bacillus cereus, Bacillus licheniformus, Streptococcus pneumonia, Helicobacter pylori, Mycobacterium tuberculosis, Staphylococcus aureus, Staphylococcus epidermis, Enterobacter faecalis, Pseudomonas syringae, Pseudomonas aeruginosa, and Bordatella pertusis.

21. A method as claimed in claim 16 in which the composition comprises a pharmaceutically acceptable carrier or excipient.

22. A method as claimed in claim 16 in which the composition is a dentifrice.

23. A method as claimed in claim 16 in which the composition is a cleaning composition.

24. A method as claimed in claim 16 in which the composition comprises at least one compound selected from the group consisting of

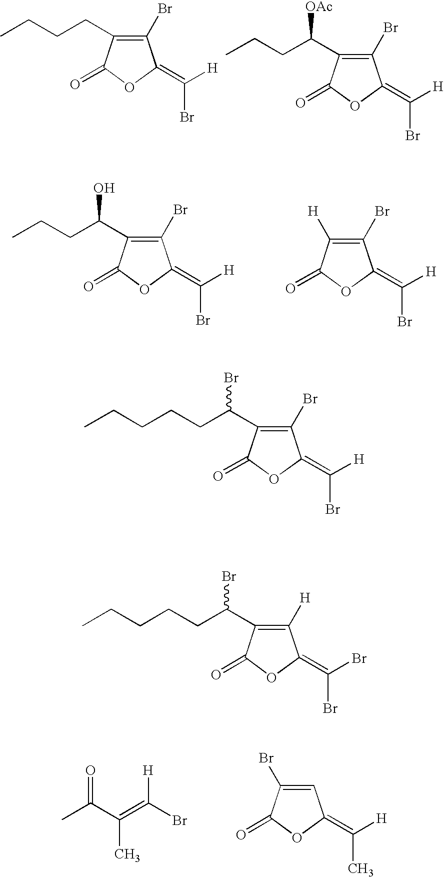

25. A method of preventing or reducing biofilm formation on a surface, the method comprising applying to the surface a composition as claimed in claim 1.

26. A method of treating bacterial infection or decreasing the severity of symptoms of bacterial infection in an animal, the method comprising administering to the animal an effective amount of the composition as claimed in claim 6.
